(19) United States
(12) Patent Application Publication
(10) Pub. No.: US 2004/0111055 A1
(43) Pub. Date: Jun. 10, 2004

(54) End effector for needle-free injection system

(76) Inventor: Keith K. Daellenbach, Portland, OR (US)

Correspondence Address: KOLISCH HARTWELL, P.C., 520 S.W. YAMHILL STREET, SUITE 200, PORTLAND, OR, 97204, US

(21) Appl. No.: 10/642,348

(22) Filed: Aug. 15, 2003

Related U.S. Application Data
(63) Continuation-in-part of application No. 10/085,564, filed on Feb. 26, 2002.

(51) Int. Cl. A61M 5/30

(52) U.S. Cl. 604/68; 604/121

(57) ABSTRACT

A needle-free injection system is described. The needle-free injection system may be suitable for injection into internal organs. In one embodiment, the needle-free injection system includes an elongate end effector that may or may not include a curved or angled distal end. The needle-free injection system may include one or more orifices.

### CROSS-REFERENCE TO RELATED APPLICATION

This application is a continuation-in-part of U.S. patent application Ser. No. 10/085,564, filed Feb. 26, 2002, which is hereby incorporated by reference.

### BACKGROUND OF THE INVENTION

The present invention provides an end effector for a needle-free injection system.

Needle-free injection systems provide an alternative to standard fluid delivery systems that typically use a needle adapted to penetrate the outer surface of a target. Typically, needle-free injection systems are designed to eject the fluid from a fluid chamber with sufficient pressure to allow the fluid to penetrate the target to the desired degree. For example, common applications for needle-free injection systems include delivering intradermal, subcutaneous, and intramuscular injections into or through a recipient's skin. For each of these applications, the fluid must be ejected from the system with sufficient pressure to allow the fluid to penetrate the tough exterior dermal layers of the recipient's skin.

It would be of great use to deliver precise quantities of fluid into an internal organ, for example during surgical procedures. Past use of needle-free delivery methods for internal organs has been limited to non-penetrating applications, where a substance is applied to or sprayed on the outside surface of an organ. However, many applications require that a fluid actually penetrate partially or completely through the internal organ.

Use of present needle-free injection systems for these applications is problematic because the design of present needle-free injection systems typically does not enable the user to effectively reach a target area that may be difficult to contact due to the recipient's physiology. Furthermore, as described above, present needle-free injection systems are typically designed to eject the fluid with sufficient pressure to penetrate the outer dermal layer. Because dermal layers are typically much tougher than the soft tissue at the external surface of an internal organ, use of an injection system that generates enough pressure to penetrate the outer dermal layer of the recipient on an internal organ might destroy at least some if not all of the functionality of the organ.

The present invention provides a needle-free injection system adapted to safely and effectively deliver a fluid into an internal organ.

### SUMMARY OF THE INVENTION

In one embodiment, the invention provides a needle-free jet injection device for delivering a fluid into an internal organ. The device includes a rigid end effector including a plurality of orifices, a fluid reservoir in fluid communication with the end effector, and an ejection mechanism adapted to eject the fluid in the fluid reservoir through the end effector and out of the orifices with sufficient pressure to penetrate the organ while preserving functionality of the organ.

In another embodiment, the invention provides an end effector for a needle-free injection device adapted to inject a fluid into an internal organ while maintaining functionality of the organ. The end effector includes a rigid elongate shaft with a plurality of orifices through which the fluid may be ejected.

In another embodiment, the invention provides a kit for performing needle-free injections into an internal organ while maintaining functionality of the organ. The kit includes a needle-free jet injection device adapted to eject a fluid, a power source for the needle-free jet injection device, and an end effector including a rigid elongate shaft including a plurality of orifices. The end effector is adapted to mate with the needle-free jet injection device such that the fluid is ejected through the orifice.

In yet another embodiment, the invention provides a method for delivering a fluid into an internal organ of a living organism. The method comprises inserting a first part of a needle-free injection system having a rigid end effector including a plurality of orifices into a living organism's body such that an internal organ within the body is contacted by at least some of the orifices in the needle-free injection system, maintaining a second part of the needle-free injection system outside of the body, and injecting a fluid through the orifices and into the internal organ such that the fluid penetrates the organ without destroying the functionality of the organ.

### BRIEF DESCRIPTION OF THE DRAWINGS

FIG. 1 is a side elevation view of one embodiment of the needle-free injection system of the present invention.

FIG. 2 is a side elevation view, partially sectioned, of another embodiment of the needle-free injection system of the present invention.

FIG. 3 is a side elevation view of one embodiment of the end effector of the present invention.

FIG. 4 is a side elevation view, partially sectioned, of one embodiment of the end effector of the present invention.

FIG. 5 is a close-up, partially sectioned, side elevation view of the distal end of the end effector of FIG. 4 taken along axis 5.

FIG. 6 is a perspective view of one embodiment of the needle-free injection system of the present invention being used to inject a fluid into an internal organ.

FIG. 7 is a close-up, fragmentary side elevation view of the distal end of another embodiment of the end effector, showing the array of orifices in two rows.

FIG. 8 is a close-up, fragmentary side elevation view of the distal end of another embodiment of the end effector, showing the array of orifices in three rows.

FIG. 9 is a view of an embodiment of the needle-free injection system of the present invention being used to inject a fluid into a prostate gland.

### DETAILED DESCRIPTION OF THE PREFERRED EMBODIMENTS

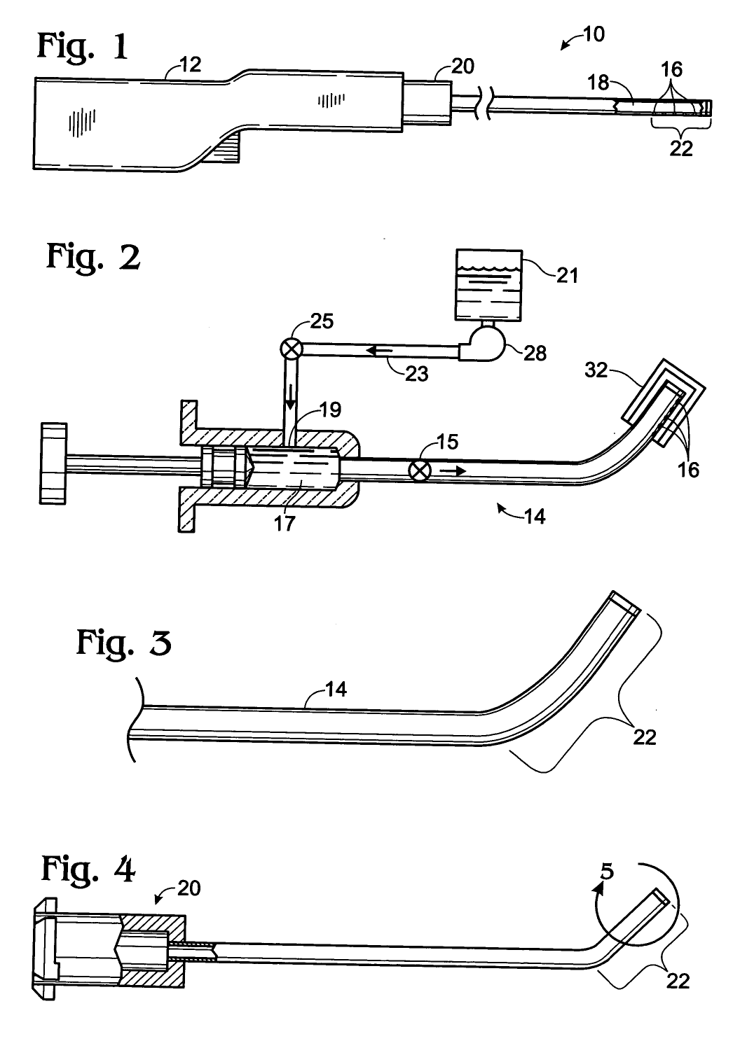

The present invention provides a needle-free injection system adapted to safely and effectively deliver a fluid into an internal organ. One embodiment of the present invention is shown in FIG. 1. The needle-free injection system 10 includes an injector 12 and an extension portion or end effector 14 having a plurality of orifices 16. Injector 12 may be, for example, a jet injection device. Typically, injector 12 will house an injection chamber adapted to receive a fluid and deliver the fluid to the plurality of orifices 16 via end effector 14. In order to prevent undesirable air bubbles in the fluid, the orifices are typically located in the distal-most portion of end effector 14. Any air bubbles in the system can be removed with a priming step.

End effector 14 may be permanently or removably attached to injector 12. For example, if the system is to be used only for internal surgical procedures, it may be desirable for the end effector to be integrated as a permanent part of the injector. Alternatively, it may be desirable to provide an injector 12 for which an optional and removable end effector 14 may be provided in order to create a more versatile system that allows the user to utilize the system for a number of different applications including both internal and transdermal applications.

A suitable jet injection device for the present invention is a modified Biojector® 2000 (B2000) needle-free injection system (Bioject, Inc., Portland, Ore.), see also U.S. Pat. Nos. 5,383,851, 5,399,163 and 5,520,629 each of which is incorporated by reference in its entirety for all purposes. The B2000 includes an outer casing which houses a replaceable carbon dioxide (CO₂) cartridge as its power source. In the commercially available product, the B2000 uses a disposable needle-free syringe including a plunger and a plastic fluid passage terminating in a nozzle. Fluid is housed within the fluid passage until the CO₂ cartridge is triggered by the user, typically by depressing or activating a button or trigger on the outside of the casing. The CO₂ cartridge releases a predetermined amount of pressurized CO₂, which pushes the plunger through the fluid passage, forcing the fluid out the nozzle with a predetermined pressure to penetrate the desired target area.

Other needle-free injectors are described, for example, in co-assigned U.S. Pat. Nos. 4,596,556, 4,790,824, 4,940,460, 4,941,880, 5,064,413, 5,312,335, 5,312,577, 5,466,220, 5,503,627, 5,649,912, 5,893,397, 5,993,412, 6,096,002, 6,132,395, 6,264,629, and 6,319,224, each of which is incorporated by reference in its entirety for all purposes.

An injector similar to those described above could be modified such that the outer casing is attached to or receives end effector 14. For example, in the case of the B2000, the disposable syringe is replaced with end effector 14. Furthermore, the plunger from the needle-free syringe could be incorporated into the end effector such that when the CO₂ cartridge is activated, the plunger forces the fluid through passage 18 in end effector 14 and out of the plurality of orifices 16.

Typically, injection system 10 will be reloadable. Of course, in a disposable or one-time use system, reloading is not required, and such systems are contemplated by the scope of the present invention. However, if it is desirable to reload the injector, this may be done through the use of a removable injection chamber, which may be refilled or which may be replaced after one or more uses.

Alternatively, as shown in FIG. 2, the injector may include an injection chamber 17, which has an opening 19 through which fluid may be loaded, providing a reloadable system capable of producing multiple injections. The end effector 14 may further include a valve 15, which, in the closed position, allows preferential back filling of the injection chamber 17 from a reservoir 21. In the open position, valve 15 allows fluid to pass through end effector 14 and then out the plurality of orifices 16. As shown, reservoir 21 may be external to the injector. Alternatively, reservoir 21 may be housed within the injector body. As shown, a fluid channel 23 may connect reservoir 21 with injection chamber 17. Fluid channel 23 may further include a valve 25 and a pump or piston 28 to enable reservoir 21 to fill injection chamber 17 when valve 15 is in the open position.

As a further alternative, the injector may include a cap 32 rather than valve 15, which, when secured to the distal end of end effector 14, blocks orifices 16 and allows preferential back filling of injection chamber 17 from reservoir 21. The user may remove cap 32 prior to injection to enable the fluid to pass through end effector 14 and out orifices 16.

The use of reservoir 21 as described above provides the injector with the ability to disperse multiple injections without requiring the injector to be reloaded. For example, after each injection, a predetermined dose of fluid could be delivered from the reservoir to injection chamber 17 in order to prime the injector for a subsequent injection. Alternatively, the system could provide for variable dose size by providing a mechanism to allow the user to adjust the amount of fluid drawn from reservoir 21 into chamber 17.

As a further alternative, rather than utilizing a separate fluid reservoir, injection chamber 17 could act as both a fluid reservoir and an injection chamber. To deliver the drug, the motion of the plunger could be controlled by means of a mechanical escapement. System 10 would be pressurized and the escapement would permit the plunger to move in single dose increments each time the escapement is toggled. This embodiment may be particularly suitable when a fixed-dose size is desirable.

As a further alternative, a motor-driven stop could be used in place of the escapement, and a clamping mechanism could be used to hold the plunger rod of injector 12. System 10 would be pressurized as above. The clamping mechanism would hold the plunger rod and prevent its movement. The motor-driven stop would move to a position appropriate for the selected dose. This movement could be either under manual or programmed control. The clamp would then capture the rod and the process would be repeated for subsequent doses. Any suitable positioning method could be used to position the stop. This system could further provide for variable dose size by providing a mechanism to allow the user to adjust the movement of the motor-driven stop.

The above-described examples are intended to be exemplary and non-limiting. Those of skill in the art will be aware of other suitable mechanisms for achieving multiple dosing capabilities, and such mechanisms are contemplated by the scope of the present invention. As will be appreciated, the ability to be used as a “repeater” allows the system to deliver multiple injections to multiple injection sites without the time-consuming need to refill or reload the injector between different injections.

As previously stated, for applications in which the intention is to inject into an internal organ, the pressure produced by system 10 and the diameter of each orifice 16 must be adjusted so as not to destroy the functionality of the organ. As will be appreciated, the soft tissue of an internal organ is less durable than the tough outer dermal layers and thus, the pressure produced by system 10 for internal organ injection applications is typically measurably less than that produced for intradermal, subcutaneous, or intramuscular injection applications. Furthermore, the appropriate system pressure and orifice diameter may be influenced by the size of the recipient. For example, it will be appreciated that the pressure required to produce a transmural injection (an injection in which the fluid has penetrated throughout the entire wall thickness of the organ) in the heart of an elephant will be significantly greater than that required to produce a transmural injection in the heart of a mouse.

Thus, system 10 may be designed to produce a range of ejection pressures that can be adjusted by the user. The B2000 may be modified to adjust the ejection pressure by changing a poppet valve spring to one with a different spring constant (k) or by modifying the degree of compression on the existing poppet valve spring. This spring resists the pressure created by the release of CO₂ from the CO₂ cartridge. The degree of resistance is determined by the desired reduction in ejection pressure. Alternatively, other suitable mechanical or electrical methods of adjusting the ejection pressure may be employed.

End effector 14 may include an adapter region 20, which connects end effector 14 to injector 12. Furthermore, end effector 14 may include a distal region 22. Distal region 22 may be angled relative to the rest of end effector 14 (as shown in FIG. 4), curved (as shown in FIG. 3), or otherwise shaped to enable the user to deliver injections to more inaccessible regions of the recipient's anatomy.

In the embodiment depicted in FIG. 4, distal region 22 has been angled relative to the rest of end effector 14. The relative angle of the distal end may vary upon the desired use of the injection system, but typically is between 30 and 90 degrees and for some embodiments is preferably around 45 degrees relative to the long axis of end effector 14.

In one embodiment, the end effector 10 may measure between four and ten inches, and preferably about six inches, from the end of the adapter and fluid reservoir to the beginning of the curvature or bend of the distal end. The distal end may measure between 0.50 and 2.00 inches, and preferably about 0.75 inches in length. Furthermore, the end effector is typically about 0.1 to 0.3 inches in diameter, and preferably about 0.2 inches in diameter. As will be appreciated, the size of end effector 14 and distal region 22 may be dependent upon the size of the intended recipient. The above ranges have been found to be appropriate for internal organ applications for use with medium-sized, i.e., 50-60 pound dogs. It is believed that such ranges may also be appropriate for use on humans. Because of internal anatomical cavity requirements, however, it will be appreciated that a longer end effector with a larger diameter may be desirable when performing internal organ injections on larger mammals, such as elephants, and a shorter end effector with a shorter diameter may be desirable when performing internal organ injections on smaller mammals, such as mice. Likewise, the size and shape of distal region 22, as well as other components of system 10, may be altered accordingly. Depending on anatomical requirements and concomitant needs for diversity of the array of multiple orifices 16, the distal region 22 of the end effector 14 may be of a cross section other than circular, for instance, it may be square or rectangular in shape.

Furthermore, the size of the adapter and fluid reservoir will, of course, depend upon the measurements of the injector that will receive the end effector. However, in some embodiments, the adapter and fluid reservoir will have an outside diameter of approximately 0.625 inches and a length of approximately 1.7 inches.

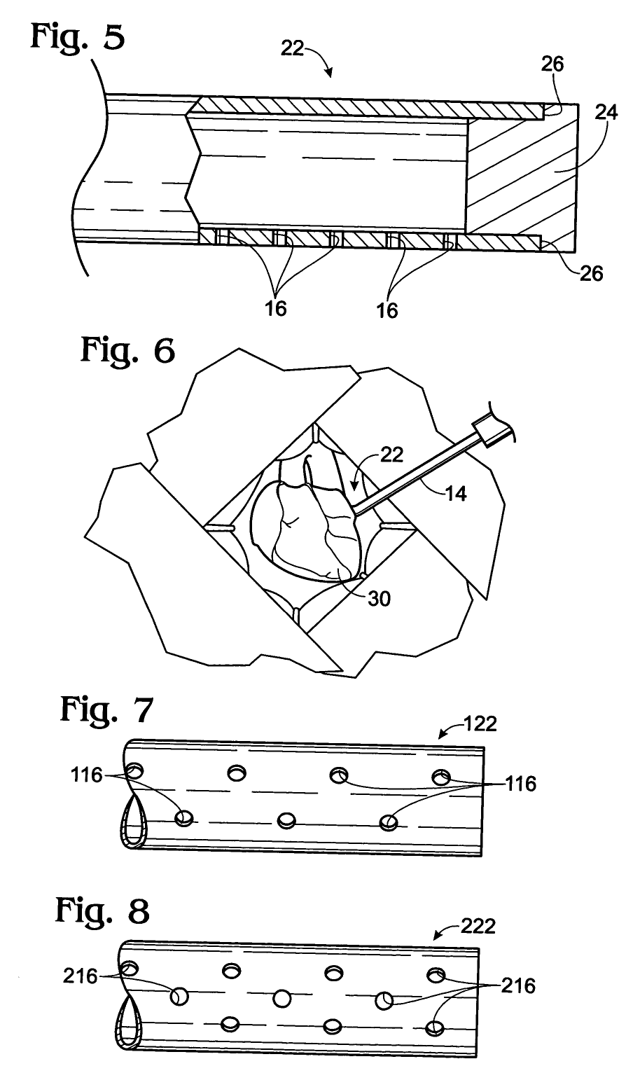

FIG. 5 is a close-up of distal region 22 of end effector 14 shown in FIG. 4, taken along axis 5. The multiple orifices 16 may be aligned along the length of the distal portion. However, alternative patterns and arrangements of the orifices are contemplated by the present invention including rings, rows, offset rows, arrays, and the like. FIG. 7 schematically depicted a pattern of two rows of orifices 116 in a distal portion 122. FIG. 8 schematically depicts a distal portion 222 having three rows of orifices 216. In another embodiment, which is not shown, one or more rows of orifices may alternatively radiate out from the central longitudinal axis of the end effector so as to be arranged around the outer circumference of the distal end of the end effector. It will be appreciated that the suitable orifice pattern may be determined based on the particular application for which the injector is to be used. For example, in many applications, it is important that the injectate be dispersed in a uniform pattern without gaps between the various injection sites, thus, arrangements that provide uniform dispersal patterns, such as offset rows, may be desired. Moreover, the number of orifices may likewise vary depending on the intended application, but typically, system 10 will include at least 1 and as many as 20 orifices.

Moreover, the diameter of the orifices may vary, both from injection system to injection system and within the same injection system. For example, the size of the orifices may differ depending upon the particular application or recipient to which the injection system is to be applied. Alternatively, differently sized orifices may be used in the same injection system to produce a particular desired injection pattern and depth.

As shown, the distal portion typically terminates in an end cap 24. The distal end of the end effector may include a lip 26 adapted to receive the end cap 24. As stated above, to minimize the production of bubbles by the injection system, the distal-most orifice in distal portion 22 should be as close to the internal edge of end cap 24 as possible. For example, to ensure a successful prime of the end effector 14 in a system having the measurements described above, it may be desirable for the distal-most orifice to be less than 0.05 inches from the internal edge of end cap 24.

Typically, end effector 14 is made of a material capable of being sterilized and able to withstand the pressure generated by injector 12. Moreover, in some laparoscopic and thoracoscopic surgical procedures, it may be desirable for end effector 14 to be rigid. In this case, suitable materials include stainless steel, titanium, composite structures of metal and plastic, and the like. If surgical procedures require a malleable and/or manipulatable end effector 14 to form around or within anatomical structures, such as contemplated with some laparoscopic, thoracoscopic, and arthoscopic procedures, plastic materials including polyurethane, high-density polyethylene, amorphous polyamide, polyetherimide, and polypropylene may be suitable.

Furthermore, it may be desirable for system 10 to be adapted to receive power from a remote power source, such as an external CO₂ tank. In this case, system 10 may include an external power source attached to an injector/end effector combination. In some cases, the external power source need not be much bigger than the end effector alone. The triggering mechanism may be located on either the power source or the injector/end effector combination.

Example—Chemical Ablation of Cardiac Tissue

The following provides an example of a suitable application for the needle-free injection system of the present invention.

The right atrium, one of the heart's four chambers, is a lower pressure pump in comparison to the ventricle. The right atrium receives unoxygenated blood from the body and passes it to the right ventricle. Because it operates at a low pressure, its wall thickness is relatively thin when compared to that of the ventricle. For example, in a dog weighing between 50 and 60 pounds, the wall thickness of the atrium is typically only about 5 millimeters, while the wall thickness of the ventricle is typically about 15 millimeters, or three times as thick.

The heart's so-called “pacemaker” is located in the sinuatrial node (SA node). This node discharges electrical impulses through pathways in the myocardium, or heart wall, to other locations of the heart, setting the entire rhythm for the cardiac cycle. If the SA node is damaged or diseased, the heart may experience sinus arrhythmias or atrial tachyarrhythmias caused by stray electrical signals being transmitted between different chambers of the heart. Chronic atrial arrhythmias can lead to dizziness, light-headedness, and loss of consciousness. In severe cases, they may lead to cardiac arrest and death.

Treatment of atrial arrhythmias includes using drugs to terminate or prevent the onset of the arrhythmia in the atrial myocardium by altering the electrical ionic fluxes across the cardiac cell membrane. Anticoagulant drugs, like DuPont Pharmaceutical's Coumadin®, are often used as an additional drug therapy in patients with chronic atrial arrhythmia to help prevent blood clotting, which can lead to stroke. Artificial pacemakers implanted into the chest cavity have electrodes attached to the external cardiac right atrial and ventricle surfaces and can help to regulate cardiac rhythm.

Other treatment methods include the intentional creation of scar tissue in the heart's myocardium. Scar tissue reduces the continuous myocardial surface near the SA node and can actually attenuate or block the propagation of the electrical impulses, thereby precluding the atrial arrhythmia. One method for forming scar tissue is the so-called “maze procedure” which involves a surgical technique that cuts through the myocardium to create a series of linear scars on the right atrium that, in effect, forms a maze pattern.

A related procedure involves a transcatheter approach in which a catheter is snaked through the femoral artery, into the left ventricle, pierced through the intraventricular septum, or wall, into the right ventricle, and then up into the inside of the right atrium. Once in the right atrium, an electrode-tipped catheter discharges energy. This hyperthermic procedure ablates the myocardium from the endocardial or inside surface of the heart and can create directed scar tissue. Like the surgical maze procedure, strategically placed scar tissue is electrically inert and disrupts the pathways for pathological atrial tachyarrhythmias.

The first hyperthermic energy source used for transcatheter ablation was a direct current (DC) power source that delivered a spark or tiny explosion damaging the endocardium. However, this method tended to leave patchy lesions and was, therefore, a less than ideal treatment method. Alternating current (AC) radio frequency (RF) and microwave energy sources have largely replaced DC energy sources.

In the transcatheter approach, the RF technique cauterizes the tissue by heating and desiccating the endocardium leading to cell necrosis and eventual development of scar tissue. However, this RF technique typically requires that the electrode-catheter be held against the endocardium for between one and five minutes per linear lesion formation and the technique often does not sufficiently produce transmural scaring because of the “heat sink” effect the blood flowing over the tip of the electrode-catheter tip may have. Furthermore, the ability to locate the catheter for application of the RF energy to a stable contact surface inside the beating heart can be difficult.

RF and microwave energy sources can also be applied to the endocardial surface of a heart that is temporarily not beating, such as during an open heart surgical procedure for mitral valve replacement. In this procedure, the beating of the heart is temporarily stopped and, with the endocardial surface of the heart exposed, an electrode-catheter tip is applied to heat the myocardial tissue leading to cell necrosis and scar tissue formation. The RF procedure has some risk of grounding the applied electrical current to the esophagus posterior to the heart which may perforate it causing a serious surgical complication. Microwave energy sources have relative less risk for this complication, however, both methods are time consuming and require an invasive open chest procedure followed by the temporary stoppage of the beating heart and open heart surgery.

Chemical ablation creates linear patterns of scar tissue that block electrical pathways by the introduction of a toxic chemical such as hypertonic saline, ethanol or formaldyhyde. However, introduction of the toxic chemical by a traditional needle and syringe tends to either deposit the chemical in a very limited area, i.e. only at the pin point injection site as a bolus of drug, or result in spraying of the chemical on the heart's surface, which typically fails to create transmural scars, which are most effective in blocking electrical pathways. An approach using a traditional needle and syringe would be slow and would have a high chance of causing undesireable point bleeding at the surface of the heart. Needle-free injection systems have the advantage of dispersing fluid into a larger area of tissue mass. Additionally, it may be possible to quickly apply the chemical ablation agent through the epicardial surface of a still beating heart during a thoracoscopic procedure which could potentially eliminate the need for an open chest surgical procedure altogether.

### Experiment I

In order to study the effectiveness of chemical ablation of cardiac tissue using a needle-free injection system, a Biojector® 2000 (B2000) needle-free injection system was modified to have a peak pressure average of 1030 psig and 2001 psig. A standard No. 2 syringe was used with the modified B2000.

A dog weighing between 50 and 60 pounds was anesthetized, the chest cavity opened, and the pericardium opened and drawn aside to expose the beating heart. Seven injections of 0.10 mL of 15% hypertonic saline and a small percentage of a methyl blue dye were made in the right atrium with the modified B2000. Nearly all of the injections left a small blue entrance mark in the whitish and fibrous epicardium. Each entrance mark was marked with a single suture. During the procedure it was unknown if the injection was transmural or if it perforated through the endocardium of the atrium. In one case an approximately 0.8 inch intradermal spacer was used. Two more shots were made into the thicker left ventrical at 1030 psig and 2001 psig.

The animal was sacrificed and the heart excised. Small sections of the myocardium were immediately stained. Some of the injections passed all the way through the myocardium into the inside of the atrium. Transmural lesions were noted at some, but not all, injection sites. However, all injections showed some dispersion in the myocardium as evidenced by necrotic tissue.

### Experiment II

To further test the use of a needle-free injection system and chemical ablation, multiple in vivo spot atrial lesions were created on the atrium of six canines. The needle-free injection systems were adjusted to eject at 643 psig, and 0.2 cc of 100% ethanol was injected into the epicardial surface. Dispersion of the fluid into the atrial myocardium took less than one second. After ablation, the animals were allowed to survive for two hours. The tissue was stained with tetrazolium tetrachloride (TTC) to assess atrial wall thickness and lesion depth.

In total, 27 atrial lesions were created. Three of the lesions caused pin-point bleeding (<0.5 cc total blood volume) that ceased spontaneously. Four of the lesions were through epicardial fat. Eighty-one percent (22) of the lesions were transmural with an average tissue depth of 2.88±1.23 mm (range 1.68 mm-6.02 mm), which was not significantly different than the non-transmural lesions (3.35±1.13 mm, p<0.05). All of the lesions through epicardial fat were transmural.

### Experiment III

In order to improve the effectiveness of chemical ablation of cardiac tissue using a needle-free injection system, a Biojector® 2000 (B2000) needle-free injection system was modified by attachment of a rigid end effector. The prototype end effector was about nine inches long, round in profile and included a tip angled at 45 degrees. The tip included three 0.0047″ diameter orifices spaced approximately 0.150″ apart in a linear arrangement. The rigid end effector was created from machined stainless steel, brass connectors with PTFE tape, and steel car break lining about 0.250″ in outer diameter. The B2000 system was further modified to have an average peak pressure (n=6) of 1011 psig and an average rise time to peak of 1.6 msec to prevent destruction of tissue due to the injection.

A 50-60 pound dog was anesthetized and attached to intravenous fluids. The dog's chest cavity was opened to allow access to the heart. The beating heart was exposed and the pericardial tissue opened and moved aside to allow direct access to the epicardium. In preparation for an electro-physical (EP) data collection effort, leads were placed to “pace” the heart, i.e. to apply an electrical potential to the epicardium, and to measure electrical signals on the other side of the pulmonary vein.

One hundred percent ethanol was injected directly into the epicardial surface of the beating heart. The heart's posterior region was reached by aid of the angled end 22 of end effector 14, as shown in FIG. 6, where the heart is shown at 30. The injections were performed in a ring around the pulmonary vein entering the left atrium.

Immediately after injection, the epicardium at the injection sites looked blanched. Following the procedure, the animal was sacrificed and the heart was excised and sectioned. Sections including ablated tissue were placed in a tetrazolium tetrachloride (TTC) solution for several minutes to stain the viable tissue a dark burgundy or red and the ablated tissue a yellowish white. Initial pathology results indicated that about half the injection sites showed transmural ablation and the other half showed partial ablation.

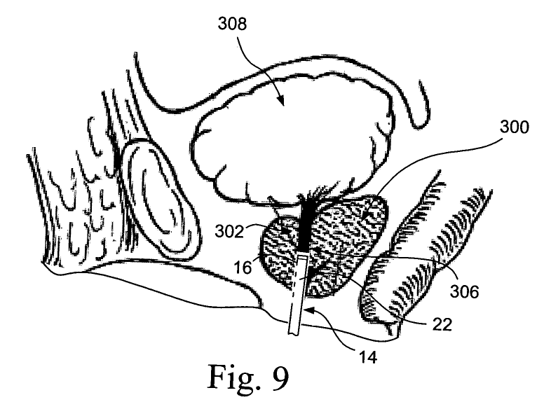

In addition to the above examples, it should be appreciated that the embodiments of the present invention may be adapted for use in a wide range of other settings. In particular, the various embodiments may be employed in connection with providing injections to internal organs other than the heart, and/or to other tissues and materials. FIG. 9 depicts use of an injection system according to the present description to deliver an injection to a prostate gland. Referring specifically to the figure, a prostate gland is shown at 300. Urethra 302 includes a penile section (not shown) and a prostatic section 306 leading through the prostate to bladder 308.

As shown in the figure, distal region 22 of end effector 14 may be positioned within prostatic section 306 of urethra 302. This positioning may be achieved by inserting distal region 22 into the urethra through the end of the penis until the distal region is in the position shown in the figure. Typically, distal region is provided toward the end of a rigid elongate shaft that extends away from the injector. In configurations adapted to be inserted through the penile portion of the urethra, it typically will be desirable that the shaft be rigid and straight, though it may be appropriate in some settings for the distal region to be slightly angled relative to the rest of the shaft. Even where an angle is used, the angle between the distal region and the rest of the shaft typically will be less than the angle used in the cardial ablation example above. Once the distal region is positioned as described and shown in the figure, the needle-free injection device may then be operated to provide an injection into prostate 300 via orifices 16. As in the above examples, the injection pressure typically is selected so as to penetrate into the prostate without destroying its functionality.

### Conclusion

The present invention provides a needle-free injection system adapted to deliver a fluid into an internal organ without destroying the functionality of the organ. It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower or equal in scope to the original claims, are also regarded as included within the subject matter of the inventions of the present disclosure.

What is claimed is:

1. A needle-free jet injection device for delivering a fluid into an internal organ, the device comprising:
a rigid end effector including at least one injection orifice, the end effector being adapted to be positioned within a prostatic section of a patient's urethra adjacent the patient's prostate gland;
a fluid reservoir in fluid communication with the end effector; and
an ejection mechanism adapted to eject the fluid from the fluid reservoir through the end effector and out of the injection orifice with sufficient pressure to penetrate the prostate gland while preserving functionality of the prostate gland.

2. The device of claim 1, wherein the rigid end effector includes a plurality of injection orifices.

3. The device of claim 2, wherein the end effector includes a straight shaft section and a distal section.

4. The device of claim 3, wherein at least some of the injection orifices are located in the distal section.

5. The device of claim 4, wherein all of the injection orifices are located in the distal section.

6. The device of claim 1, wherein the ejection mechanism is further adapted to allow the device to eject multiple doses of fluid without refilling the fluid reservoir.

7. The device of claim 1, wherein the fluid includes ethanol.

8. The device of claim 2, wherein at least some of the injection orifices are arranged linearly along the length of the end effector.

9. The device of claim 1, wherein the injection orifices are arranged in multiple rows along the length of the end effector.

10. The device of claim 9, wherein the rows are offset from each other.

11. A needle-free jet injection device for delivering a fluid into an internal organ, the device comprising:
a fluid reservoir;
an extension structure adapted to be inserted within a patient's urethra so that a distal region of the extension structure is positioned adjacent the patient's prostate gland, wherein the distal region of the extension has an at least partially hollow interior that fluidly communicates with the fluid reservoir; and
an ejection mechanism adapted to eject the fluid from the fluid reservoir through the extension structure and out of a plurality of injection orifices provided in the distal region of the extension structure.

12. The device of claim 11, wherein the ejection mechanism is adapted to eject the fluid with sufficient pressure to penetrate the prostate gland while preserving functionality of the prostate gland.

13. The device of claim 11, wherein the fluid includes ethanol.

14. The device of claim 11, wherein at least some of the injection orifices are arranged linearly along the length of the extension structure.

15. The device of claim 11, wherein the injection orifices are arranged in multiple rows along the length of the extension structure.

16. The device of claim 15, wherein the rows are offset from each other.
